(19) United States
(12) Patent Application Publication
(10) Pub. No.: US 2021/0055243 A1
(43) Pub. Date: Feb. 25, 2021

(54) Gas sensor and related methods for detecting the vapor phase of electrochemically-active substances

(71) Applicant: Nathan Charles Schattke, Yorkville, IL (US)

(72) Inventor: Nathan Charles Schattke, Yorkville, IL (US)

(21) Appl. No.: 16/873,935

(22) Filed: Aug. 18, 2020

Related U.S. Application Data
(60) Provisional application No. 62/922,605, filed on Aug. 19, 2019.

(51) Int. Cl. G01N 27/12; G01N 1/24; G01N 33/497

(52) U.S. Cl. CPC G01N 27/122; G01N 2001/245; G01N 33/497; G01N 1/24

(57) ABSTRACT

A gas sensor for detecting extremely low levels of electrochemically-active substances by cumulative absorption of the substances on a reactive medium, such as for detecting analyte emitted by bed bugs. A fan provides a gas flow path for moving room air mixed with an analyte from bed bugs into the sensor and toward a plurality of electrodes disposed in the sensor. An electrolytic membrane is disposed on an active electrode area of each of the plurality of electrodes. When at least one of the plurality of electrodes determines that the concentration of the analyte reaches a predetermined detection limit, the electrical conductivity of at least one electrode changes, and circuitry which is in communication with the electrodes provides an output signal indicative of the presence of bed bugs in the room.

### CROSS REFERENCE TO RELATED APPLICATION

The present application is related to, and claims priority of, U.S. provisional patent application Ser. No. 62/922,605, filed on Aug. 19, 2019, which is incorporated herein in its entirety.

### FIELD OF THE INVENTION

The present invention relates generally to a gas sensor and related methods for detecting extremely low levels of electrochemically-active substances by cumulative absorption of the substances on a reactive medium. While many potential applications or uses of the sensor exist, one such use includes detecting the presence of bed bugs (also commonly known as “bedbugs”).

### BACKGROUND OF THE INVENTION

Bed bugs (Cimex lectularius) are cryptic insects which cause significant economic and emotional damage. Bedbugs are a particular nuisance in the lodging industry where the frequent turnover of residents provides ample opportunity for new infestations unknowingly carried in the contents of the resident's luggage. Hotels do not want to have their reputation besmirched by any disclosures of bed bug infestations. Consequently, valuable resources are necessarily utilized annually in the detection, and in the eradication, of bed bugs.

Typically, three onsite prior art routes are used for the detection of bed bugs: visual, traps or trained animals. Of course, visual inspection is often inadequate since bed bugs are known to hide particularly well. Traps and trained dogs have also met with mixed success.

Because bed bugs only emerge in the dark, it is difficult to find and eradicate them. The visibility problem was solved in the twentieth century with aerosol insecticides, particularly with DDT. However, with DDT falling out of favor and with humans traveling more widely, bed bugs are carried from habitation to habitation. With the use of powerful pesticides curtailed indoors, bed bugs are resurging around the world. Our challenge now is to apply insecticides judiciously. Ideally, insecticides are applied only where bed bugs actually exist, and early before the bed bugs feed, mate and colonize since larger infestations are more difficult to eradicate. There is thus a need for an accurate and sensitive bed bug detector.

Since bed bugs emit a vapor with a characteristic odor, detecting bed bugs scents and vapors is a practical approach.

Several methods have been utilized in laboratory experiments to detect the amounts and types of chemicals which are emitted by bed bugs. None of these methods are commercially practical for field use by professionals. However, these experiments do show that there is a known suite of chemicals which are related to bed bug infestations.

### SUMMARY OF THE INVENTION

Accordingly, it is a principal object of the present invention to provide apparatus and methods for more accurately detecting the presence of bed bugs.

Another object of the present invention to provide apparatus which detects the presence of bedbugs by sensing chemicals emitted by the bed bugs.

A further object of the present invention is to provide such apparatus treated with a reactive chemical for absorbently sensing the presence of bed bugs which may be inexpensively replaced after completing a test and before initiating a new bed bug test.

A still further object of the present invention is to provide a membrane with sensing electrodes apparatus which is inexpensive and economical to replace.

Yet another object of the present invention is to present the methods used to select an electrolyte from available materials, targeted to the analytes for a wide range of low level, long term vapor emitters.

These and other objects will become apparent to those skilled in the art in light of the following disclosure and accompanying drawings.

The present invention relates generally to a gas sensor for detecting extremely low levels of electrochemically-active substances by cumulative absorption of the substances on a reactive medium. One such use for this sensor is to detect the presence of bed bugs (also commonly known as “bedbugs”). However, other uses may exist, such as in the pollution remediation field, particularly with regard to those pollutants in the vapor phase. In those situations, there will be different analytes and enclosure conditions. For example, if the methods of the present invention are used to detect tritium in a well, the well would be the enclosure.

The apparatus and methods utilize a fan to provide a gas flow path for moving room air mixed with an analyte from bed bugs toward a plurality of electrodes disposed in the sensor. An ionic membrane is disposed on an active electrode area of each of the plurality of electrodes The plurality of electrodes thereby monitors the target gasses. When the concentration of the analyte on the membrane reaches a predetermined detection limit, the electrodes become conducive and circuitry which is in communication with the electrodes provides an electrical signal or indication of the presence of bed bugs in the room.

Another aspect of the present invention is that the ionic membrane is treated with a reactive material. The electrodes may be printed with an ionic membrane to provide a membrane which is inexpensive and which permits a one-time use of the membrane. The electrodes are connected to a circuit for control and interpretation and to report to an end user, including remotely.

An important aspect of the present invention is the selection of the absorbent membrane on the electrodes, as well as the electrode material, to provide a selective absorption of the analytes in a situation needing detecting, such as bed bugs, as well as detecting the changes created by that analyte. The absorbent system must selectively collect the most unique chemical or chemical class associated with the target, then exhibit an ionic change detectable via a change in the electrical conductivity of the electrode. That change can be a resistive, capacitive or inductive (RCL) change. That change can also be a change in cyclic current draw to sweeping voltage (CV) excitations. One such absorbent material is the ionic liquid butyltrimethylammonium methanesulfonate (B3MS).

The present invention also includes methods, including the steps of providing a gas sensor, utilizing a fan to provide a gas flow path for moving room air mixed with an analyte into said gas sensor, disposing an ionic membrane in said gas sensor, disposing a plurality of electrodes on said membrane, said electrodes having an active sensing area for monitoring the target gasses, said plurality of electrodes providing a change in output signal when the concentration of the analyte reaches a predetermined detection limit. A further step includes communicating the change in output signal from at least one of the electrodes to logic circuitry to inform the user that detection of analytes has occurred. Thus, the gas sensor detects extremely low levels of target gasses by cumulative chemisorption on a reactive medium.

In accordance with another aspect of the invention, the electrode surfaces may be functionalized with catalytic metals such as platinum, palladium, iridium, the lanthanide groups; and/or with functional groups such as allyl, acetate, or borate. An example of a useful electrode surface is carbon base functionalized with allyl modified platinum nanoparticles.

With any instrument system there is a cost to sensitivity line that is exponential on the axises and linear; the more sensitive, the more expensive. The present invention can move the cost way down for very faint analytes due to the selective reaction absorption on an engineered electrolyte.

### BRIEF DESCRIPTION OF THE DRAWINGS

The present invention, together with the further objects and advantages thereof, may best be understood by reference to the following description taken in conjunction with the accompanying drawings, in the figures, in which like reference numerals identify like elements, and in which:

FIG. 1 is a block diagram illustrating a bed bug detection system in accordance with the present invention;

FIG. 2 is a diagrammatic view of a membrane assembly which may be utilized in the bedbug detection system shown in FIG. 1, also in accordance with the present invention;

FIG. 3 illustrates a diagrammatic view of an electrode which may be utilized in the membrane assembly shown in FIG. 2;

FIG. 4 illustrates a chart or table with a ranked list of test chemicals;

FIG. 5a illustrates a chemical diagram for an ionic liquid 1,2-DiMethyl-3-PropylImidazolium (PDMI) or 1-Propyl-2,3-DiMethylImidazolium TetraFluoroBorate (DMPI BF4);

FIG. 5b illustrates a chemical diagram for an ionic liquid 1-Benzyl-3-MethylImidazolium TetraFluoroBorate (BzMI BF4 or simply Bz);

FIG. 5c illustrates a chemical diagram for an ionic liquid ButylTriMethylImidazolium MethaneSulfonate (B3MA MS); and

FIG. 5d illustrates a chemical diagram for an ionic liquid TetraEthylAmmonium TetraFluoroBorate (TEA BF4).

### DESCRIPTION OF THE PREFERRED EMBODIMENTS

In accordance with the invention, a gaseous sensor is able to detect extremely low levels of electrochemically-active substances by cumulative absorption of the substances on a reactive medium. Engineered ionic liquids can be specified and functionalized to detect many different chemical analytes. This is achieved at low cost and with high sensitivity and selectivity, as will be appreciated in view of the further disclosure below.

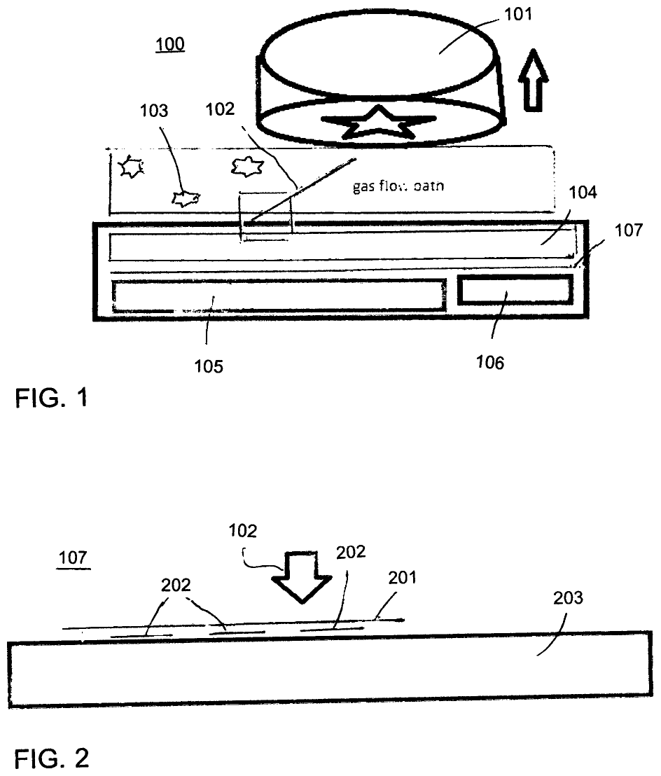

While many potential applications and/or uses exist for such a gaseous sensor, in accordance with one such application or use, the sensor may be utilized in the form of a bed bug detection system, generally designated by numeral 100 in FIG. 1. System 100 is typically employed where bed bugs may be present. Such locations may include, for example, apartments, hotel or motel rooms, dormitory rooms, laundry or cleaning facilities, bedding storage rooms, linen closets and the like. These locations are hereinafter collectively referred to as “room” or “rooms”.

System 100 includes a fan 101 for stirring the air and to create a gas flow path 102 into the system 100. Preferably, the fan is selected to agitate target compounds which may be heavier than air and yet in the gas or vapor phase. For example, if bed bugs are present, there may be analyte 103 from bed bugs mixed in with the room air. The gas flow path 102 carries the analyte 103 to interact with one or more of the electrodes 202 shown in FIG. 2.

A potentiostat circuit 104 conducts interrogation of the plurality of electrodes 202. A logic circuitry module 105 controls the potentiostat circuit and provides an output signal when detection of the target gas occurs. An input/output module 106 receives the output signal from the logic circuitry module 105 and provides an output signal for actuating an alarm, display or indicator (not shown) to provide audio and/or visual confirmation when detection of a target gas occurs.

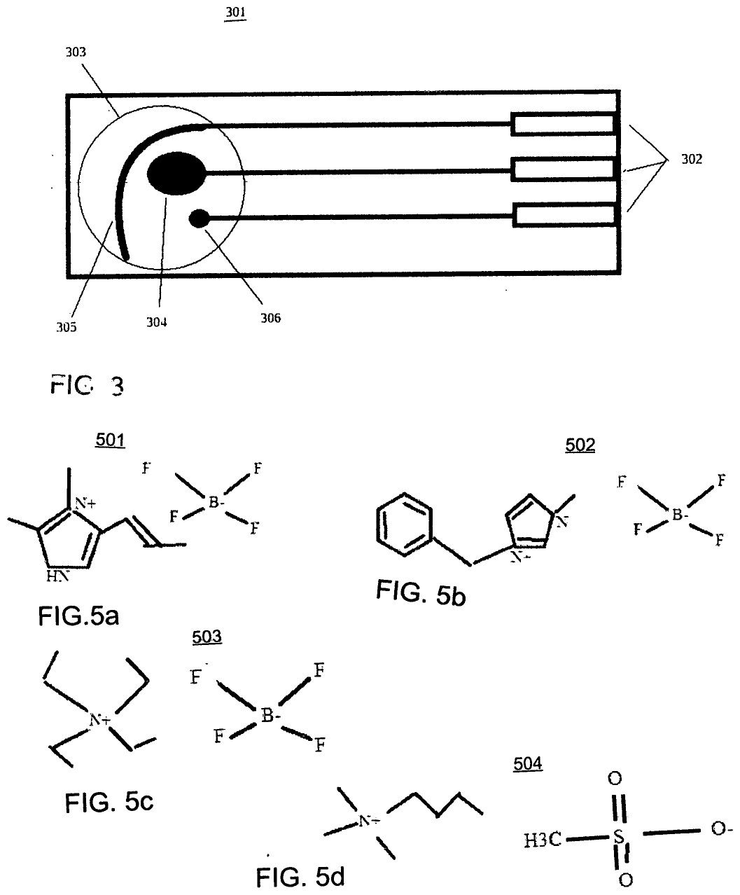

Disposed partially or entirely within system 100 is a membrane assembly 107, which is further described with reference to FIG. 2. As discussed in FIG. 1, gas flow path 102 provides room air including any analyte present in the gas flow path to the membrane assembly 107. Membrane assembly 107 preferably consists of an ionic liquid-based membrane 201, a plurality of electrodes 202 and a substrate 203. While three electrodes are shown in FIG. 2, it will be appreciated that more or less may be utilized depending upon the application and/or design requirements. The electrodes 202 may be printed with the ionic membrane 201 to provide a membrane which is inexpensive and which permits a one-time use of the membrane. That is, electrodes 202 may be removed and replaced for a subsequent use or test by the system 100. The electrodes are connected to circuitry for control and interpretation and to report to an end user, including remotely. An example of the electrodes 202 is shown in FIG. 3.

Shown in FIG. 3 is an electrode, generally designated by reference numeral 301, which can be utilized in the present invention. For example, electrode 301 is commercially available from Pine Instruments of Durham, N.C. under component designation RRPE1001C. Electrode 301 has three electrically conductive tabs 302 for connection to the potentiostat circuit 105. An active electrode area (within circle 303 of electrode 301) is coated with the ionic liquid mix. The enlarged end 304, connected to one of the tabs 302, is the working electrode for driving current to the ionic liquid. The curved end 305, connected to another of the tabs 302, is the counter electrode for receiving the current from enlarged end 304. End 306, connected to another tab 302, is a reference electrode and both the working and counter voltages are measured in relation to the reference voltage. Some embodiments may, alternatively, connect the counter and reference electrodes together as one electrode, and yet other embodiments may have electrode systems each with different chemistry.

Preferably, the electrodes 301 are easily removable when a sensing test has been completed, such that a new set of electrodes may be installed in the gas sensor for the next use or test. To this end, electrodes 301 may slide or snap into a connector or socket on the substrate 203. Before a new set of electrodes is installed in the gas sensor, the active electrode area 303 of each electrode needs to be treated with the preferred ionic liquid. The ionic liquid may be applied by printing, spraying, painting or dipping techniques.

Gas sensor 100 may be fabricated to be quite compact, such as about pocket sized for convenience of the user. If the size of the population of bed bugs in the room is significant, the sensor 100 can quickly determine their presence, for example, in less than an hour. Preferably, the electronics of the sensor 100 are battery powered such that the sensor is conveniently portable for placement wherever needed.

The gas sensor 100 may also be known as a biosensor since it detects faint chemical signatures such as those emitted by ectoparasites, particularly bed bugs, employing an absorptive membrane-based potentiostat based system to identify specific semiochemicals. This nanosensor device uses an absorptive membrane or fluid and an electrochemical signal to detect bed bug chemicals. Thus, an important and unique aspect of the present invention is the liquid. The most desirable liquid is one that demonstrates the strongest change in resistance or current draw for the narrowest molecular range within a wide electrochemical window, thereby enabling reliable readings across a wide range of potentials under low vapor pressure, thereby allowing for long absorption times without evaporation. Ionic liquids as a class are suitable for this application being the most chemically-selective, responsive and robust.

By having a very low vapor pressure, long scan times are possible leading to sampling of larger spaces with accuracy. This allows for sector by sector scanning before individual area scans. For example, a hallway on a cruise ship may be scanned to see if individual cabins need scanning.

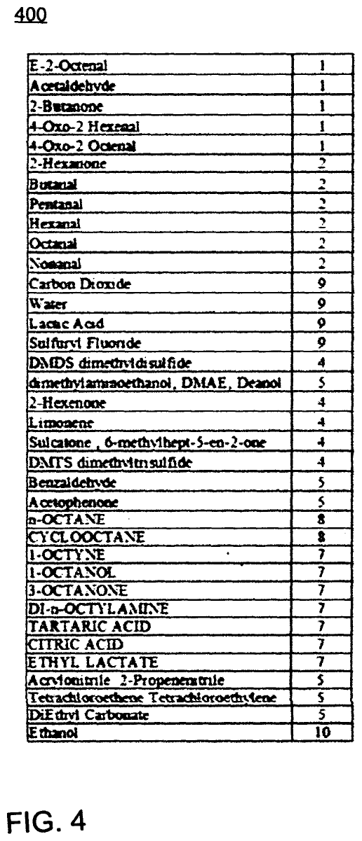

The table 400 of FIG. 4 shows a ranked list of test molecules. This list can be created by those skilled in the art of desirable analytes, and undesirable interferents, to compare against potential electrolytes. A rank of 1 is most desirable and a rank of 10 is least desirable. Strong negative changes in Gibbs Free Energy (GFE) of association with analytes and positive GFE with interferents, with the proposed electrolyte, can be found via computational chemistry tools. These findings can then be verified with physical measurements.

Ionic liquid absorbs the bed bug indicator molecules. Ionic liquids have a very low vapor pressure which allows for very long absorption times without evaporation. The ionic liquid also selectively maximizes interactive energy between vapor analyte chemicals and the liquid, and minimizes energies for non-analyte chemicals. The changed chemistry of the liquid on the membrane changes the resistance or other electrochemical behavior proportional to the concentration of the analyte. This change of resistance at one or more of the electrodes results in a change in a signal to a microprocessor. The microprocessor reads the signal from the electrodes and transmits the results to a human-readable display device.

The selection of the absorbent membrane on the electrodes, as well as the electrode material, is important to provide a selective chemisorption of the analytes emitted by bed bugs as well as detecting changes created by that analyte. The absorbent system must selectively collect the most unique chemical or chemical class associated with the target, then exhibit an ionic change detectable via the electrode. That change can be a resistive, capacitive or inductive (RCL) change. That change can also be a change in cyclic current draw to sweeping voltage (CV) excitations.

The ionic liquids which are preferred for creating the absorbent membrane, specifically for bed bugs, are shown by their chemical symbols in FIGS. 5a-5d. Namely, 1,2-DiMethyl-3-PropylImidazolium (PDMI) or 1-Propyl-2,3-DiMethylImidazolium TetraFluoroBorate (DMPI BF4) 501, 1-Benzyl-3-MethylImidazolium TetraFluoroBorate (BzMI BF4 or simply Bz) 502, ButylTriMethylImidazolium MethaneSulfonate (B3MA MS) 503, and TetraEthylAmmonium TetraFluoroBorate (TEA BF4) 504.

The electrode surfaces may also be functionalized with catalytic metals such as platinum, palladium, iridium, the lanthanide groups; and/or with functional groups such as allyl, acetate, or borate. An example of a useful electrode surface is carbon base functionalized with allyl modified platinum nanoparticles.

While detection of analytes emitted by bed bugs is a preferred embodiment of the present invention, it will be appreciated that the system 100 is also useful in detecting all electro-chemically active substances present in an air flow. For example, yet other uses may be in detecting stinkbugs and in detecting cancer.

In view of the disclosure above, the present invention also includes methods, including the steps of providing a gas sensor, utilizing a fan to provide a gas flow path for moving room air mixed with an analyte into said gas sensor, disposing an ionic membrane in said gas sensor, disposing a plurality of electrodes on said membrane, said electrodes having an active sensing area for monitoring the target gasses, said plurality of electrodes providing a change in output signal when the concentration of the analyte reaches a predetermined detection limit. A further step includes communicating the change in output signal from at least one of the electrodes to logic circuitry to inform the user that detection of analytes has occurred. Thus, the gas sensor detects extremely low levels of target gasses by cumulative chemisorption on a reactive medium.

While particular embodiments of the invention have been shown and described, it will be apparent to those skilled in the art that changes and modifications may be made thereto without departing from the invention in its broader aspects.

1. A gas sensor for detecting extremely low levels of electrochemically-active substances by cumulative absorption of the substances on a reactive medium, said gas sensor comprising:
a fan to provide a gas flow path for moving room air mixed with the electrochemically-active substances into said gas sensor,
at least one electrode disposed in said sensor, said electrode having an active sensing area, and
a reactive medium disposed on the active sensing area of said at least one electrode for monitoring the air mixed with the electrochemically-active substances, said at least one of electrode providing a change in electrical conductivity when the concentration of the electrochemically-active substances on the reactive medium reaches a predetermined level.

2. The gas sensor in accordance with claim 1 wherein the change in conductivity of said at least one electrode is translated into an output signal indicative of a confirmation of the presence of electrochemically-active substances in the air.

3. The gas sensor in accordance with claim 2 further comprising a display in communication with the output signal to display the results of the monitoring of the air.

4. The gas sensor in accordance with claim 1 wherein the reactive medium disposed on the active area of said at least one electrode consists of an ionic liquid.

5. The gas sensor in accordance with claim 4 wherein the ionic liquid is selected from a group consisting of 1,2-DiMethyl-3-PropylImidazolium (PDMI) or 1-Propyl-2,3-DiMethylImidazolium TetraFluoroBorate (DMPI BF4), 1-Benzyl-3-MethylImidazolium TetraFluoroBorate (BzMI BF4 or simply Bz), ButylTriMethylImidazolium MethaneSulfonate (B3MA MS), and TetraEthylAmmonium TetraFluoroBorate (TEA BF4).

6. The gas sensor in accordance with claim 1 wherein the electrochemically-active substances consist of analytes emitted by bed bugs.

7. The gas sensor in accordance with claim 1 wherein said at least one electrode consists of a plurality of electrodes.

8. A method for detecting extremely low levels of electrochemically-active substances by cumulative absorption of the substances on a reactive medium, said method including the steps of:
providing a gas sensor,
utilizing a fan in the gas sensor to provide a gas flow path for moving room air mixed with the electrochemically-active substances into said gas sensor,
disposing at least one electrode in said sensor, said electrode having an active sensing area, and
disposing a reactive medium on the active sensing area of said at least one electrode for monitoring the air mixed with the electrochemically-active substances, said at least one of electrode providing a change in electrical conductivity when the concentration of the electrochemically-active substances on the reactive medium reaches a predetermined level.

9. The method for detecting extremely low levels of electrochemically-active substances by cumulative absorption of the substances on a reactive medium in accordance with claim 8 comprising the additional step of translating the change in conductivity of said at least one electrode into an output signal indicative of a confirmation of the presence of electrochemically-active substances in the air.

10. The method for detecting extremely low levels of electrochemically-active substances by cumulative absorption of the substances on a reactive medium in accordance with claim 9 further comprising the step of using the output signal to display the results of the monitoring of the air.

11. The method for detecting extremely low levels of electrochemically-active substances by cumulative absorption of the substances on a reactive medium in accordance with claim 8 including the step of using an ionic liquid for the reactive medium disposed on the active area of said at least one electrode, the ionic liquid being selected by energy optimized tools compared with available materials.

12. The method for detecting extremely low levels of electrochemically-active substances by cumulative absorption of the substances on a reactive medium in accordance with claim 11 including the step of selecting the ionic liquid from a group consisting of 1,2-DiMethyl-3-PropylImidazolium (PDMI) or 1-Propyl-2,3-DiMethylImidazolium TetraFluoroBorate (DMPI BF4), 1-Benzyl-3-MethylImidazolium TetraFluoroBorate (BzMI BF4 or simply Bz), ButylTriMethylImidazolium MethaneSulfonate (B3MA MS), and TetraEthylAmmonium TetraFluoroBorate (TEA BF4).

13. The method for detecting extremely low levels of electrochemically-active substances by cumulative absorption of the substances on a reactive medium in accordance with claim 8 wherein the electrochemically-active substances consist of analytes emitted by bed bugs.

14. The method of claim 8 for selecting electrolytes with matching properties needed for detection of very low levels of analytes at costs much lower than previous methods.

15. The method for detecting extremely low levels of electrochemically-active substances by cumulative absorption of the substances on a reactive medium in accordance with claim 8 wherein said at least one electrode consists of a plurality of electrodes.
